(19) United States
(12) Patent Application Publication
(10) Pub. No.: US 2014/0107500 A1
(43) Pub. Date: Apr. 17, 2014

(54) METHODS AND APPARATUS TO DETERMINE VENTILATORY AND RESPIRATORY COMPENSATION THRESHOLDS

(71) Applicant: BreathResearch, (US)

(72) Inventors: Charalampos Christos Stamatopoulos, Athens (GR); Panagiotis Giotis, Berlin (DE); Nirinjan Bikko Yee, Walnut Creek, CA (US)

(73) Assignee: BREATHRESEARCH, Walnut Creek, CA (US)

(21) Appl. No.: 13/830,818

(22) Filed: Mar. 14, 2013

Related U.S. Application Data
(60) Provisional application No. 61/610,976, filed on Mar. 14, 2012.

(51) Int. Cl. A61B 5/0205

(52) U.S. Cl. CPC A61B 5/0205
USPC 600/484

(57) ABSTRACT

An apparatus and method for determining a ventilator threshold (VT) and respiratory compensation threshold (RCT) is disclosed. The method comprises measuring breathing patterns of a user. It also comprises measuring a heart rate of the user. Finally, it comprises determining a VT and a RCT for the user based on an analysis of the breathing patterns and the heart rate.

### CROSS-REFERENCE TO RELATED APPLICATIONS

This application claims priority from U.S. Provisional Application No. 61/610,976 Attorney Docket BRTH-0001.A, filed Mar. 14, 2012, assigned to the assignee of the present application and the entire disclosure of which is incorporated herein by reference.

### FIELD OF THE INVENTION

Embodiments according to the present invention generally relate to methods and apparatus for determining a user's ventilatory threshold (VT) and respiratory compensation threshold (RCT) and, more particularly, to methods and apparatus for determining a user's VT and RCT using an electronic device.

### BACKGROUND OF THE INVENTION

To determine an athlete's VT and RCT, a complex medical device (often referred to as gas or metabolic analyzers) and the personnel to conduct the test are required. This is often cost prohibitive. One additional scientific way to measure VT and RCT is to use a blood lactatate analysis. However, this is an invasive medical procedure. Another method to measure VT and RCT is to use the Foster talk test, but, in this case, the athlete has too much room for personal and subjective interpretation and thus the results may not be as reliable as the more scientific methods.

### BRIEF SUMMARY OF THE INVENTION

Accordingly, there is a need for improved methods and apparatus to determine VT and RCT. Using the beneficial aspects of the systems described, without their respective limitations, embodiments of the present invention provide novel solutions to the challenges inherent in determining VT and RCT in a non-invasive and accurate fashion.

In one embodiment, a method for determining a ventilatory threshold (VT) and respiratory compensation threshold (RCT) is disclosed. The method comprises measuring breathing patterns of a user. It also comprises measuring a heart rate of the user. Finally, it comprises determining a VT and a RCT for the user based on an analysis of the breathing patterns and the heart rate.

In one embodiment, an apparatus for determining a ventilatory threshold (VT) and respiratory compensation threshold (RCT) is disclosed. The apparatus comprises a memory comprising an application for determining a VT and a RCT stored therein. It also comprises a microphone for recording a breathing of a user and a communicative interface for receiving pulse rate information from the user. Further, it comprises a processor coupled to the memory, wherein the processor is configured to operate in accordance with the application to: (a) measure breathing patterns of the user using the breathing recorded using the microphone; (b)measure heart rate of the user using the pulse rate information; and (c) determine a VT and a RCT for the user based on an analysis of the breathing patterns and the heart rate. In one embodiment, the apparatus may be portable.

In another embodiment, a computer-readable storage medium having stored thereon, computer executable instructions that, if executed by a computer system cause the computer system to perform a method to determine a ventilator threshold (VT) and respiratory compensation threshold (RCT) is disclosed. The method comprises measuring breathing patterns of a user. It also comprises measuring a heart rate of the user. Finally, it comprises determining a VT and a RCT for the user based on an analysis of the breathing patterns and the heart rate. In one embodiment, the computer system may be a portable electronic device.

The following detailed description together with the accompanying drawings will provide a better understanding of the nature and advantages of the present invention.

### BRIEF DESCRIPTION OF THE DRAWINGS

Embodiments of the present invention are illustrated by way of example, and not by way of limitation, in the figures of the accompanying drawings and in which like reference numerals refer to similar elements.

FIG. 1 shows one example of a pulse measuring device for a mobile electronic device according to an exemplary embodiment of the present invention.

FIG. 2 shows another example of a pulse measuring device for a mobile electronic device according to an exemplary embodiment of the present invention.

FIG. 3 shows an exemplary breathing microphone set-up used in the methods and apparatus of the present invention.

FIG. 4 shows electronic apparatus running software to determine VT and RCT according to an exemplary embodiment of the present invention.

In the figures, elements having the same designation have the same or similar function.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a mobile device application that uses a microphone as a means for recording the user's breathing for the purpose of measuring the VT and RCT thresholds. The microphone can periodically listen to breath sounds at the mouth and the software automatically derives estimates of VT and RCT therefrom. The mobile application may include one or more computer implemented procedures that can record breath sounds and receive pulse rate information from the user to generate an estimate of VT and RCT.

An electronic device, such as a portable computer, mobile electronic device, or a smartphone, may be configured with appropriate software and inputs to permit breath sound data recording and recording data from a heart rate monitor simultaneously.

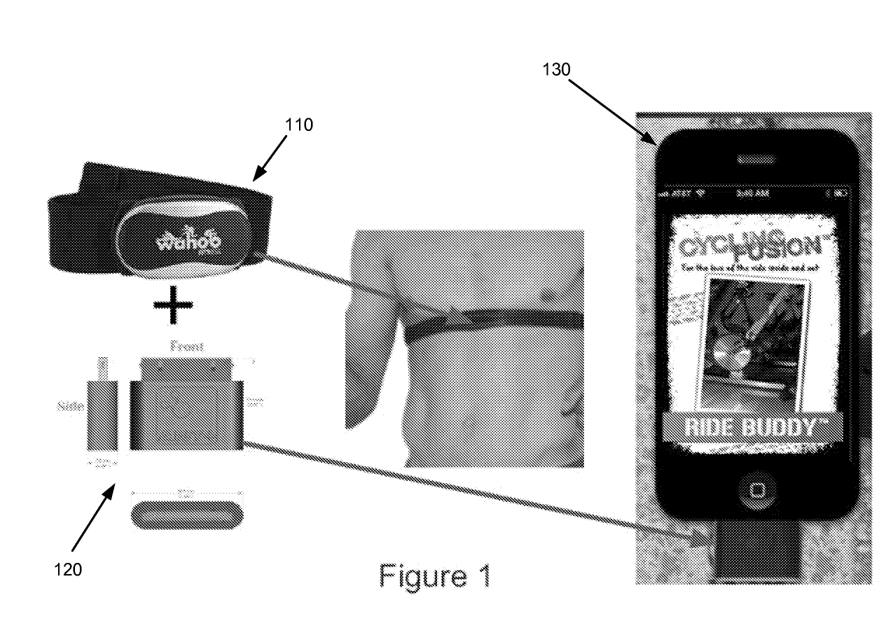

FIG. 1 shows one example of a pulse measuring device for a mobile electronic device according to an exemplary embodiment of the present invention. The pulse measuring device shown in the embodiment illustrated in FIG. 1 is a heart monitor transmitter belt 110 that is communicatively coupled with a receiver module 120. The transmitter 110 transmits heart rate information, among other things, to the receiver module 120. In one embodiment, the transmission can take place wirelessly using a near field communication protocol such as Bluetooth. The receiver module 120, in one embodiment, can plug into a portable electronic device 130 such as a smart-phone. The portable electronic device 130, in one embodiment, can use the information from the receiver module 120 to undertake further analysis of the pulse rate. Also it can use the pulse rate in conjunction with the breath sound to generate an estimate of the VT and RCT.

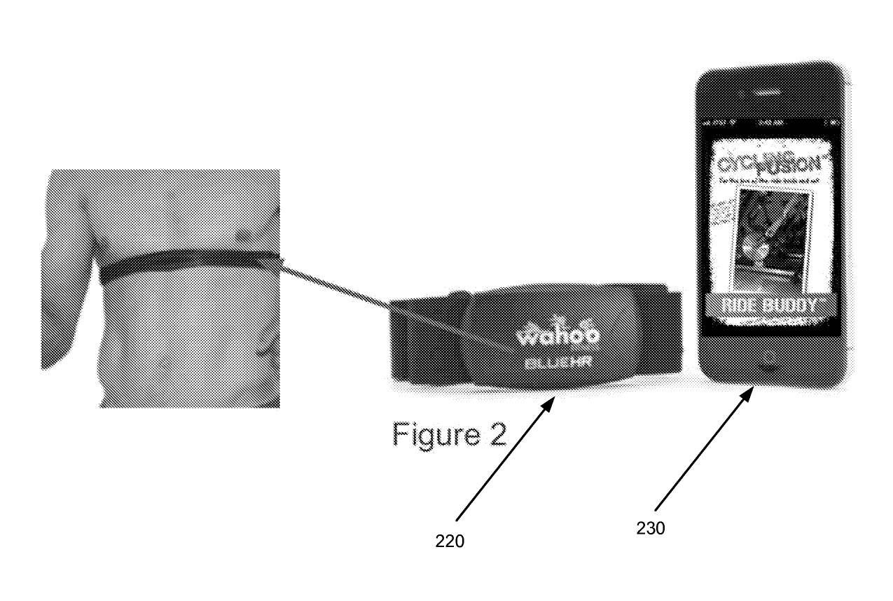

FIG. 2 shows another example of a pulse measuring device for a mobile electronic device according to an exemplary embodiment of the present invention. In the embodiment illustrated in FIG. 2, the heart monitor transmitter belt 220 is configured to transmit signals directly to an electronic device 230, such as a smart-phone. The computer-implemented procedures running on device 230 can decode the transmission to undertake further analysis of the pulse rate. Also they can use the pulse rate correlated to the VT and RCT estimates from the breath sound analysis to create heart training zones for the user. In one embodiment, the transmission can take place wirelessly using a near field communication protocol such as Bluetooth. Alternatively, in one embodiment, electronic device 230 can be at a remote location and receive the transmission through a cellular signal.

A microphone can pick up the breathing patterns of the user during exercise and a heart monitor transmitter belt, or some other heart rate monitoring device, can simultaneously pick up the heart beats and send them in a continuous (regular frequency) fashion to a heart monitor receiver.

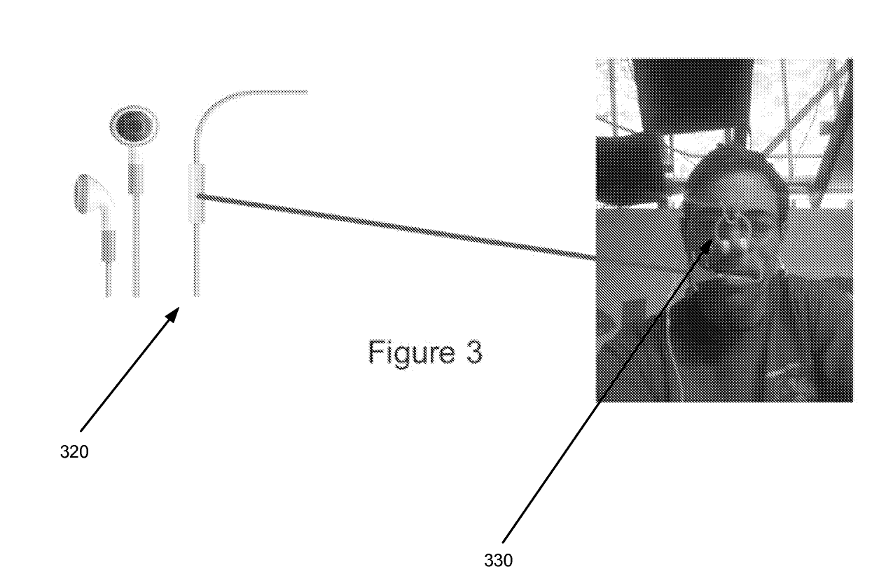

FIG. 3 shows an exemplary breathing microphone set-up used in the methods and apparatus of the present invention. In one embodiment, a conventional microphone 320, available commercially, can be used to record the breathing patterns of the user. By using only the microphone 320 that comes with many electronic devices (such as an iPad® or iPhone®) and the software as described herewithin, the present invention can provide VT and RCT data for a fraction of the cost of alternative options. Moreover, the test can be self-administered, not requiring special testing equipment or trained personnel.

Various designs may be used to create an accurate breath sound measurement. In some embodiments, as shown in FIG. 3, the user's nose may be closed to ensure the microphone at the user's mouth captures the entirety of the user's breathing.

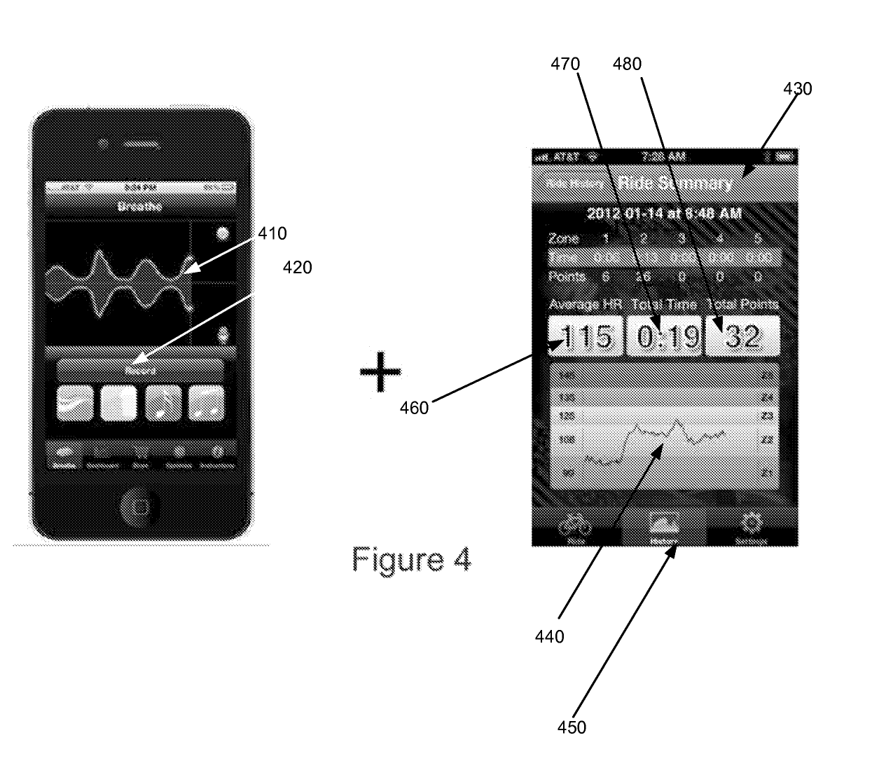

FIG. 4 shows electronic apparatus running software to determine VT and RCT according to an exemplary embodiment of the present invention.

The software can both display the breathing patterns 410 and/or heart rate values 440 on the display screen of the electronic device. It can also save the heart rates, the breathing patterns and all of its related information contained in the users breathing onto the storage medium contained in the electronic device, computer or mobile device. In one embodiment, the user can be provided with an option to start recording the breathing pattern at the click of a push-button 420.

The software can then analyze the information obtained through the breathing sound measurements in order to determine the associated ventilatory (VT) and respiratory compensation (RCT) thresholds and their respective heart rate values from the heart monitor receiver. Research can be conducted to develop a relationship between breathing patterns and VT/RCT ratio. With this information, the software may be programmed with these relationships to provide an accurate estimate of the user's VT and RCT.

The software may be written in one or more computer programming codes and may be stored on a computer readable media. The software may include program code adapted to perform the various method steps as herein described.

The software could be used by itself to analyze any saved audio file that might have been taken from any recording device other than the electronic device having the microphone. If the user had a time line with heart rate values that corresponded to the saved audio file, they could use the software by itself to produce the intended result of the invention.

To use the embodiment of the invention illustrated in FIG. 1, a person would set up the electronic device 130 near the user who is exercising (typically on a stationary bike or a treadmill). They would have the user put a heart monitor 110 on their body, plug the heart monitor receiver 120 into the electronic device, and then begin the recording session by telling the software that the test has begun.

In one embodiment, the software can also collect and save information regarding the user's workout program. As shown in FIG. 4, for example, the software could display the user's ride summary 430 after the user is done exercising on a stationary bike. The user can access the ride summary after the ride by clicking on a “History” tab 450. The display under the “History” tab of the software can be programmed to show the user's average heart rate 460, the total time of the workout 470 and total points 480 accumulated by the user. The display can also be configured to show a graphical display 440 of the user's heart rate.

Once the user confirms that the test is complete, the software can perform the required analysis to determine ventilatory (VT) and respiratory compensation (RCT) thresholds and their related heart rates in Beats Per Minute (BPM).

Embodiments of the present invention could be used in the medical field or any field where ventilatory (VT) and respiratory compensation (RCT) thresholds are used to train athletes or diagnose medical conditions.

It should be understood, of course, that the foregoing relates to exemplary embodiments of the invention and that modifications may be made without departing from the spirit and scope of the invention as set forth in the following claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as may be suited to the particular use contemplated.

Embodiments according to the invention are thus described. While the present disclosure has been described in particular embodiments, it should be appreciated that the invention should not be construed as limited by such embodiments, but rather construed according to the below claims.

What is claimed is:

1. A computer-readable storage medium having stored thereon, computer executable instructions that, if executed by a computer system cause the computer system to perform a method to determine a ventilator threshold (VT) and respiratory compensation threshold (RCT), said method comprising:
measuring breathing patterns of a user;
measuring heart rate of said user; and
determining a VT and a RCT for said user based on an analysis of said breathing patterns and said heart rate.

2. The computer-readable medium as described in claim 1, wherein said computer system comprises a microphone and wherein the measuring of said breathing patterns comprises recording breathing of said user using said microphone.

3. The computer-readable medium as described in claim 2, wherein said computer system is selected from the group comprising: a portable computer, a mobile electronic device, and a smart phone.

4. The computer-readable medium as described in claim 1, wherein said measuring of said heart rate comprises monitoring a heart beat of said user using a heart rate monitoring device.

5. The computer-readable medium as described in claim 4, wherein said heart rate monitoring device is a heart monitor transmitter belt.

6. The computer-readable medium as described in claim 1, wherein said method further comprises displaying said breathing patterns and said heart rate on a display screen.

7. The computer-readable medium as described in claim 6, wherein said method further comprises displaying statistical information regarding an exercise program during which said breathing patterns and said heart rate are measured for said user.

8. The computer-readable medium as described in claim 1, wherein said breathing patterns and said heart rate are transmitted to said computer system wirelessly.

9. The computer-readable medium as described in claim 1, wherein said analysis comprises using a programmed relationship between said breathing patterns and said heart rate to determine said VT and said RCT for said user.

10. The computer-readable medium as described in claim 9, wherein said breathing patterns and said heart rate used in said analysis are pre-recorded, and wherein said breathing pattern and said heart rate are recorded within the same time interval.

11. The computer-readable medium as described in claim 1, wherein said breathing patterns can be measured from a prerecorded audio file, wherein said audio file comprises recorded samples of breathing of said user.

12. The computer-readable medium as described in claim 1, wherein said measuring of said heart rate is performed while said user is exercising.

13. A method for determining a ventilator threshold (VT) and respiratory compensation threshold (RCT), said method comprising:
measuring breathing patterns of a user;
measuring heart rate of said user; and
using a computer system determining a VT and a RCT for said user based on an analysis of said breathing patterns and said heart rate.

14. The method of claim 13, wherein the computer system comprises a microphone and wherein the measuring of said breathing patterns comprises recording a breathing of said user using the microphone.

15. The method of claim 14, wherein said computer system is selected from the group comprising: a portable computer, a mobile electronic device, and a smart phone.

16. The method of claim 13, wherein the measuring of said heart rate comprises monitoring a heart beat of said user using a heart rate monitoring device.

17. The method of claim 16, wherein said heart rate monitoring device is a heart monitor transmitter belt coupled to said computer system.

18. The method of claim 13, wherein said method further comprises displaying said breathing patterns and said heart rate on a display screen of said computer system.

19. The method of claim 13, wherein said analysis comprises using a programmed relationship between said breathing patterns and said heart rate to determine said VT and said RCT for said user.

20. The method of claim 13, wherein said breathing patterns can be measured from a prerecorded audio file, wherein said prerecorded audio file comprises recorded samples of breathing of said user.

21. The method of claim 13, wherein said measuring of said heart rate is performed while said user is exercising.

22. The method of claim 13, wherein said method further comprises displaying statistical information regarding an exercise program during which said breathing patterns and said heart rate are measured for said user.

23. A portable apparatus for determining a ventilator threshold (VT) and respiratory compensation threshold (RCT), said apparatus comprising:
a memory comprising an application for determining a VT and a RCT stored therein;
a microphone for recording a breathing of a user;
a communicative interface for receiving pulse rate information from said user;
a processor coupled to said memory, wherein said processor is configured to operate in accordance with said application to:
measure breathing patterns of said user using said breathing recorded using said microphone;
measure heart rate of said user using said pulse rate information; and
determine a VT and a RCT for said user based on an analysis of said breathing patterns and said heart rate.
